(19) United States
(12) Patent Application Publication
(10) Pub. No.: US 2004/0215143 A1
(43) Pub. Date: Oct. 28, 2004

(54) Hollow stylet for infusion catheter systems, devices and methods

(76) Inventors: Martin Brady, Phoenix, MD (US); Christoph Pedain, Muenchen (DE)

Correspondence Address: Schwegman, Lundberg, Woessner & Kluth, P.A., P.O. Box 2938, Minneapolis, MN, 55402, US

(21) Appl. No.: 10/753,979

(22) Filed: Jan. 8, 2004

Related U.S. Application Data
(60) Provisional application No. 60/439,728, filed on Jan. 13, 2003.

(51) Int. Cl. A61M 1/00

(52) U.S. Cl. 604/122; 600/433

(57) ABSTRACT

This document discusses, among other things, systems, devices, and methods that include a hollow-tube stylet shaped and sized for insertion into a flexible infusion catheter for use as a guiding stylet during insertion of the stylet and catheter toward a subject. Fluid is loaded into the hollow tube stylet, and released upon withdrawal of the stylet from the infusion catheter. This avoids air from occupying the infusion catheter upon withdrawal of the stylet, which could disturb the infusion of the agent.

### CROSS-REFERENCE TO RELATED APPLICATIONS

This patent application claims the benefit of priority, under 35 U.S.C. Section 119(e), to Brady et al. U.S. Provisional Patent Application Serial No. 60/439,728, entitled “HOLLOW STYLET FOR INFUSION CATHETER SYSTEMS, DEVICES AND METHODS,” filed on Jan. 13, 2003 (Attorney Docket No. 00723.065PRV).

### TECHNICAL FIELD

This document relates generally to catheters, and particularly, but not by way of limitation, to a device for use in inserting a catheter into tissue.

### BACKGROUND

Catheters are thin tubes that can be inserted into a patient's body, such as to deliver therapeutic agent(s), diagnostic sensor(s), etc. to remote locations inside the body. Infusion catheters are sometimes used for infusing a therapeutic agent into tissue, such as brain tissue. Such infusion delivers the agent through the catheter directly to a specific target site within the tissue. Infusion is particularly important for delivering a substance to the brain, since a blood-brain barrier limits the effectiveness of systemic therapy by preventing large molecules from passing from the bloodstream into the brain. The infusion catheter may remain in place in the tissue for hours or days.

One typical method for placing a catheter into brain tissue for direct infusion is described below. The catheter is inserted through an access opening in the skull, and advanced from this access point in a straight trajectory, directly through the tissue, until a distal end of the catheter reaches a target location. Then, to prevent infection at the access site, a portion of the catheter remaining outside the skull is tunneled beneath the scalp to a position several centimeters away from the access site, and the skin over the access site is closed. The agent can then be introduced through the catheter tube by connecting a filled syringe or pump to a proximal end of the catheter.

Catheters used for direct infusion are often made of flexible polymer materials. This allows them to undergo sharp bends for tunneling. It also ensures that a movement of the proximal end of the tube, outside the skull, will not transfer any significant force to the distal end resting in position in the tissue at the target site. One disadvantage of such flexible material is that it is too flaccid to be pushed in a straight trajectory through the tissue to the target position.

To overcome this problem, such catheters are generally inserted using a guide wire, which is sometimes called a stylet or a trocar. Stylets are straight, rigid wires of diameter similar to or slightly less than the interior diameter of the catheters for which they are designed. They are made out of comparatively stiff materials, e.g., stainless steel or other metal(s). A typical stylet diameter is around one millimeter. The stylet is inserted into the catheter to stiffen it while it is being introduced into tissue and advanced to the target location.

After the catheter is introduced into tissue and reaches the target location, the stylet is removed from the catheter. This leaves only the catheter in place in the patient's tissue. The present inventors have recognized that one resulting problem is that removing the stylet leaves behind air filling the interior volume of the catheter. However, such air cannot easily be removed. Any fluid later introduced into the catheter for infusion will first push this remaining air into the tissue. Air infused into the tissue can adversely effect the infusion of a fluid agent. For example, bubbles of air in the tissue can cause the infusing fluid to distribute in an unpredictable and/or undesirable pattern. For these and other reasons, which will become apparent upon reading the following detailed description and viewing the drawings that form a part thereof, the present inventors have recognized an unmet need for improved catheters, catheter insertion devices, and ancillary tools and methods.

### SUMMARY

This document discusses, among other things, improved catheters, catheter insertion devices, and ancillary tools and methods for reducing or eliminating the amount of air that is introduced into the catheter interior during withdrawal of the stylet. In one example, a hollow but rigid tube, which may be referred to as a “hollow stylet”, replaces the solid, rigid guide wire. In one example, the hollow stylet has an outer diameter that is similar to an inner diameter of the flaccid hollow tubular catheter, such that no appreciable amount of air lies between the outer wall of the hollow stylet and the inner wall of the flaccid catheter. Thus, the hollow stylet can be conceptualized as completely filling the inside of the catheter tube, with a fluid completely filling the inside of the hollow stylet. In one example, a distal end of the hollow stylet is open. However, by temporarily closing a proximal end of the fluid-filled hollow stylet, fluid is discouraged from leaking out the open distal end port until the proximal end of the hollow stylet is opened, such as before it is extracted from the infusion catheter. One possible realization of the hollow stylet includes a needle cannula with an unsharpened blunt distal end hole. In this example, the needle cannula is designed such that its outer diameter fits somewhat snugly within the inner diameter of the flaccid infusion catheter. In this example, the needle cannula is configured with a length that is equal to or greater than that of the flaccid infusion catheter.

Before inserting the infusion catheter, the hollow stylet is inserted therewithin. The interior of the hollow stylet is filled with the fluid therapeutic agent, or with some other fluid, such as sterile saline. A proximal region of the hollow stylet is then sealed or otherwise closed to retain the fluid therewithin. During insertion of the infusion catheter, the hollow stylet performs the same catheter-stiffening function as a normal solid stylet. After insertion, the proximal end of the hollow stylet is unsealed or opened. Then the hollow stylet is removed from the infusion catheter. This creates a resulting vacuum at the distal end of the infusion catheter as the hollow stylet is extracted from the infusion catheter. This resulting vacuum draws the fluid out of the interior of the hollow stylet. The drawn-out fluid fills the infusion catheter interior. This reduces or eliminates the problem of air being left behind by removal of a solid stylet.

In one example, there is a seal between the interior wall of the infusion catheter and the exterior wall of the hollow stylet. This seal prevents air from entering the infusion catheter from its proximal end. In one example, the proximal end of the hollow stylet is connected to a fluid-filled tube or other reservoir. This ensures that the hollow stylet introduces enough fluid with which to fill the entire interior volume of the infusion catheter during removal of the hollow stylet. The proximal end of the infusion catheter is then connected to the infusion source carrying a therapeutic fluid agent.

### BRIEF DESCRIPTION OF THE DRAWINGS

In the drawings, which are not necessarily drawn to scale, like numerals describe substantially similar components throughout the several views. Like numerals having different letter suffixes represent different instances of substantially similar components. The drawings illustrate generally, by way of example, but not by way of limitation, various embodiments discussed in the present document.

FIG. 1 is a schematic diagram illustrating generally a partial cross-section of a portion of a system including a flexible catheter and a hollow stylet.

FIG. 2 is a flow chart illustrating generally one method of using a system that includes a flexible catheter and a hollow stylet.

FIG. 3 is a schematic diagram illustrating generally a portion of the flexible catheter and a portion of the hollow stylet, in which a proximal end of the catheter seals snugly around the hollow stylet, but more distal portions of the catheter are not as snugly fitted around the hollow stylet.

### DETAILED DESCRIPTION

In the following detailed description, reference is made to the accompanying drawings which form a part hereof, and in which is shown by way of illustration specific embodiments in which the invention may be practiced. These embodiments are described in sufficient detail to enable those skilled in the art to practice the invention, and it is to be understood that the embodiments may be combined, or that other embodiments may be utilized and that structural, logical and electrical changes may be made without departing from the scope of the present invention. The following detailed description is, therefore, not to be taken in a limiting sense, and the scope of the present invention is defined by the appended claims and their equivalents.

In this document, the terms “a” or “an” are used, as is common in patent documents, to include one or more than one. Furthermore, all publications, patents, and patent documents referred to in this document are incorporated by reference herein in their entirety, as though individually incorporated by reference. In the event of inconsistent usages between this documents and those documents so incorporated by reference, the usage in the incorporated reference(s) should be considered supplementary to that of this document; for irreconcilable inconsistencies, the usage in this document controls.

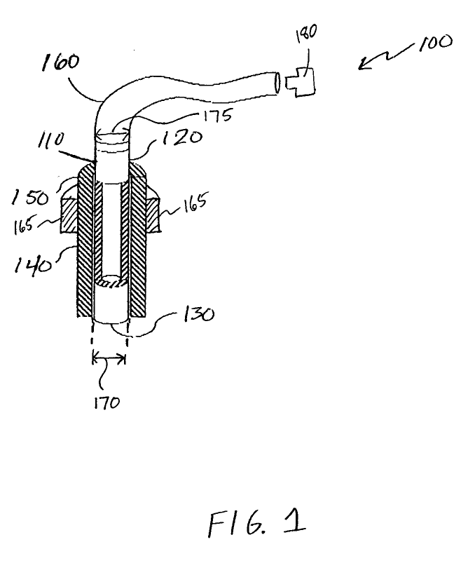

FIG. 1 is a schematic diagram illustrating generally, by way of example, but not by way of limitation, a system 100 including an elongate flexible tubular infusion or other catheter 140 and an elongate hollow stylet tube 110. In this example, the hollow tube stylet 110 is inserted into the catheter 140 for use as a stiffening guidewire to guide the catheter 140 to a target within a patient's brain or other tissue. In one example, a proximal end 120 of the hollow-tube stylet 110 is connected to a separate or integral fluid reservoir 160, which may be enclosed (e.g., a bladder) or open. This ensures that enough fluid can be provided during removal of the hollow-tube stylet 110 from the infusion catheter 140 to fill the interior of the infusion catheter. This, in turn, prevents air from entering the infusion catheter and subsequently disturbing infusion. (In one example, the reservoir is simply the same hollow-tube stylet 110, which is long enough such that it holds enough fluid to fill the lumen of the infusion catheter 140 when the hollow-tube stylet 110 is withdrawn from the infusion catheter 140). The hollow-tube stylet 110 is made of a material that is stiffer than that of the flexible catheter 140 to provide the catheter/stylet system 100 with sufficient torsional stability to allow tunneling and guiding through brain or other tissue toward a desired target.

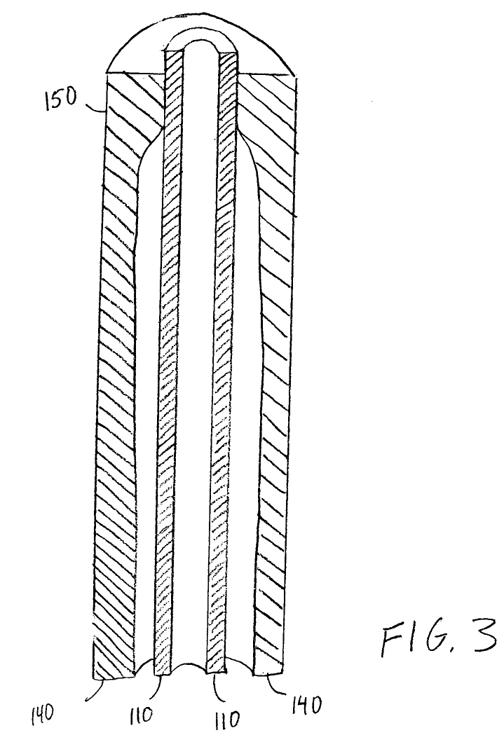

In one example, at least a proximal end 150 of the catheter 140 is optionally clamped (e.g., using encircling clamp 165) or otherwise provides an inner diameter 170 that closes snugly around the outer diameter 175 of the hollow-tube stylet 110, such as during removal of the hollow-tube stylet 110 from the catheter 140. In another example, the inner diameter 170 of the entire catheter 140 is sized to snugly seal at least the proximal end 150 of the catheter 140 to the outer diameter 175 of the hollow-tube stylet 110. In a further example, more distal portions (relative to the sealing proximal end) of the inner diameter of the catheter 140 are more loosely sized than the outer diameter of the hollow-tube stylet 110, as illustrated in FIG. 3. This allows easier sliding of the hollow-tube stylet 110 into and out of the catheter 140. FIG. 1 also illustrates an optional plug 180. A portion of the plug 180 is sized and shaped to fit within the hollow stylet 110 to temporarily seal its proximal end to retain fluid within the hollow stylet 110. The plug 180 is removed before the hollow stylet 110 is withdrawn from the catheter 140, to permit the retained fluid to be released into the interior of the catheter. Alternatively, the proximal end of the hollow stylet 110 can be temporarily sealed using a cap or clamp, (or even a gloved finger, if desired). Moreover, if pinching or clamping is used, the sealing need not take place at the proximal end of the hollow stylet, but may be performed at a more intermediate portion of the hollow stylet 110.

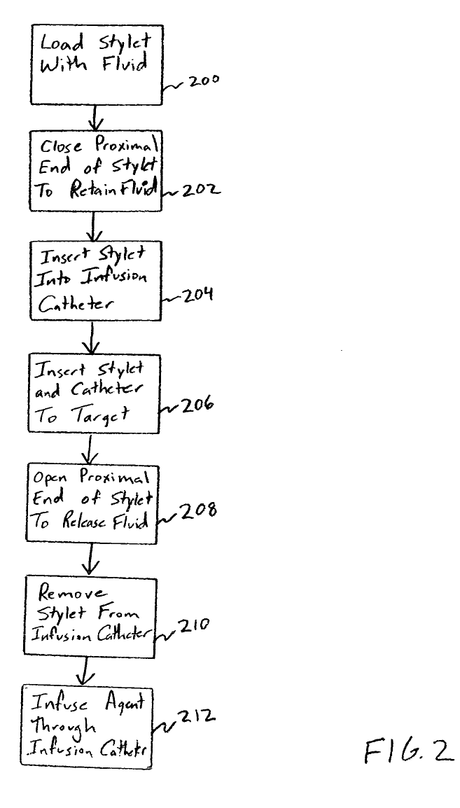

FIG. 2 is a flow chart illustrating generally, by way of example, but not by way of limitation, one method of using the system 100. In this example, at 200, the hollow-tube stylet 110 is filled with fluid. At 202, a proximal end of the hollow-tube stylet 110 is temporarily closed to retain the fluid within the hollow-tube stylet 110. At 204, the hollow-tube stylet 110 is then inserted into the infusion catheter 140. (Alternatively, the hollow-tube stylet 110 is loaded with fluid, and then its proximal end closed, after it has been inserted into the infusion catheter 140). At 206, the hollow-tube stylet 110 and the catheter 140 are inserted together to the target location within the patient's brain or other tissue. (Alternatively, the stylet is inserted first, then the catheter is inserted over the guidewire stylet). The insertion of the stylet 110 and the catheter 140 may utilize an orientable and fixable trajectory guide, in conjunction with an image-guided surgical (IGS) workstation, to aim the stylet 110 and the catheter 140 toward a desired target. At 208, the proximal end of the hollow-tube stylet 110 is opened to allow release of the fluid from therewithin. At 210, the hollow-tube stylet 110 is withdrawn from within the infusion catheter 140. The resulting vacuum draws the fluid out of the hollow-tube stylet 110 and into the infusion catheter 140, thereby preventing or avoiding air from entering and remaining within the catheter 140 and disturbing the subsequent infusion. If desired, the catheter is then secured. At 212, an agent is infused through the catheter 140 to the target location in the tissue.

In a further embodiment, the tunneling progress of the hollow-tube stylet 110 during surgery is tracked with one or more locators, such as a locator that is remotely detectable using a positioning system coupled to an image-guided surgical (IGS) workstation. In one such example, at least one remotely-detectable locator is attached to at least one of the hollow-tube stylet 110 and the catheter 140. As the hollow-tube stylet 110 and the catheter 140 are tunneled through tissue toward a desired target, the locator's progress is tracked and displayed on a monitor of the IGS workstation.

It is to be understood that the above description is intended to be illustrative, and not restrictive. For example, the above-described embodiments may be used in combination with each other. Many other embodiments will be apparent to those of skill in the art upon reviewing the above description. The scope of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects.

What is claimed is:

1. A system comprising:
a hollow tube, including a proximal end and a distal end and a lumen extending therebetween, wherein the hollow tube is shaped and sized to permit insertion into a lumen of a flexible tubular infusion catheter, and wherein the hollow tube is stiffer than the infusion catheter such that the hollow tube acts as a stylet for guiding the catheter through tissue to a target location.

2. The system of claim 1, in which the lumen of the hollow tube is filled with a fluid, and in which the proximal end of the hollow tube is configured to be closed to retain the fluid within the lumen of the hollow tube.

3. The system of claim 1, further including a fluid reservoir that is coupled to the proximal end of the hollow tube.

4. The system of claim 3, wherein the hollow tube and the fluid reservoir are sized to hold enough fluid to fill the lumen of the infusion catheter after withdrawal of the hollow tube from the lumen of the infusion catheter.

5. The system of claim 1, further including a flexible tubular infusion catheter including a proximal end and a distal end and a lumen extending therebetween, the lumen of the infusion catheter sized and shaped to permit insertion of the hollow tube therein.

6. The system of claim 5, in which the proximal end of the infusion catheter sealingly engages around the hollow tube when a portion of the hollow tube is located with the lumen of the infusion catheter.

7. The system of claim 6, in which the proximal end of the tubular catheter includes a clamp that closes around the hollow tube.

8. The system of claim 6, in which the lumen of the catheter includes a diameter having at least two different values at different locations along the lumen of the catheter.

9. The system of claim 1, in which the hollow tube includes a remotely detectable locator.

10. The system of claim 9, further including a positioning system that permits location of the locator.

11. The system of claim 10, further including an image-guided surgical workstation coupled to the positioning system.

12. The system of claim 1, further including means for temporarily sealing the proximal end of the hollow tube to retain fluid within the hollow tube.

13. The system of claim 1, further including a cap sized and shaped to cap off a proximal end of the hollow tube.

14. The system of claim 1, further including a plug sized and shaped to plug a proximal end of the hollow tube.

15. The system of claim 1, further including a clamp sized and shaped to pinch off a portion of the hollow tube.

16. A system comprising:
a flexible tubular infusion catheter including a proximal end and a distal end and a lumen extending therebetween, the lumen of the infusion catheter sized and shaped to permit insertion of the hollow tube therein;
a hollow tube, including a proximal end and a distal end and a lumen extending therebetween, wherein the hollow tube is shaped and sized to permit insertion into a lumen of the flexible tubular infusion catheter, and wherein the hollow tube is stiffer than the infusion catheter such that the hollow tube acts as a stylet for guiding the catheter through tissue to a target location; and
wherein at least a portion of an inner diameter of the flexible infusion catheter snugly seals to an outer diameter of the hollow tube to prevent air from passing therebetween as the hollow tube is withdrawn from the flexible infusion catheter.

17. The system of claim 16, in which a proximal portion of the inner diameter of the flexible infusion catheter snugly seals to the outer diameter of the hollow tube, and in which a distal portion of the inner diameter of the flexible infusion catheter more loosely encircles the outer diameter of the hollow tube than the proximal portion.

18. A method comprising:
loading a hollow-tube stylet with fluid;
inserting the stylet into a lumen of a flexible infusion catheter to provide enough stiffening to the catheter to guide the catheter through living tissue toward a target;
directing the stylet and the catheter through tissue to the target; and
withdrawing the stylet from the catheter, in which the withdrawing includes releasing the fluid from the stylet into the lumen of the catheter to avoid air from occupying the lumen of the catheter upon withdrawal of the stylet.

19. The method of claim 18, further including temporarily closing a proximal end of the stylet, after the loading the stylet with fluid, to assist in retaining the loaded fluid within the stylet.

20. The method of claim 19, further including opening the proximal end of the stylet after the inserting the stylet into the lumen of the catheter and before the withdrawing the stylet, to release fluid from the stylet into lumen of the catheter.

21. The method of claim 18, further including infusing a fluid agent through the catheter after the withdrawing the stylet.
